(19) United States
(12) Patent Application Publication
(10) Pub. No.: US 2019/0282101 A1
(43) Pub. Date: Sep. 19, 2019

(54) METHOD FOR IDENTIFYING AND DETERMINING A PHYSIOLOGICAL CHARACTERISTIC USING INVISIBLE LIGHT

(71) Applicant: SOLID YEAR CO., LTD., New Taipei City (TW)

(72) Inventor: Wei-Hsin TSAI, NEW TAIPEI CITY (TW)

(21) Appl. No.: 15/956,162

(22) Filed: Apr. 18, 2018

(30) Foreign Application Priority Data
Mar. 19, 2018 (TW) 107109190

(51) Int. Cl. A61B 5/00; A61B 5/024

(52) U.S. Cl. CPC A61B 5/0077; A61B 5/7275; A61B 5/02416; A61B 5/746; A61B 5/7465; A61B 5/7282

(57) ABSTRACT

A method for identifying and determining a physiological characteristic using invisible light includes the steps of: respectively activating at least two light-emitting units to emit an invisible light onto a human body to be detected in such a manner that only one light-emitting unit is activated at a time wherein each of the at least two light-emitting units is configured to emit a different wavelength of invisible light, then capturing a real-time image signal from the human body corresponding to the invisible light emitted by each light-emitting unit and reflected by the human body, and then analyzing each real-time signal and defining a skin area according to the analysis result, and finally calculating the change of reflected light intensity of the skin area to obtain a measured value corresponding to the physiological characteristic of the human body.

### BACKGROUND OF THE INVENTION

### 1. Field of the Invention

The present invention relates to detection device technology and more particularly, to a method for identifying and determining a physiological characteristic using invisible light.

### 2. Description of the Related Art

To more comprehensively and efficiently take care of infants and young children or patients with mobility problems and the elderly, in addition to the parents or nurses touching the person in need of care, there are many auxiliary detection tools or devices to assist in confirming the physiological state of the person being taken care of.

A simple detection device uses a digital camera to record images of the caretaker for a long period of time. The captured digital images are then transmitted over the Internet to a cloud server, computer or mobile phone so that the caregiver can observe whether the caretaker appears an abnormal state. In addition, there are monitoring methods that directly monitor the physiological signal by sticking electrodes on the caretaker, achieving the effects of detection and monitoring.

### SUMMARY OF THE INVENTION

The present invention has been accomplished under the circumstances in view. It is the main object of the present invention to provide a method for identifying and determining a physiological characteristic using invisible light, which can more accurately and instantly identify the physiological characteristic of the human body, improving the quality and efficiency of care.

To achieve this and other objects of the present invention, a method for identifying and determining a physiological characteristic using invisible light comprises firstly the step of respectively activating at least two light-emitting units to emit an invisible light onto a human body to be detected in such a manner that only one light-emitting unit is activated at a time wherein each of the at least two light-emitting units is configured to emit a different wavelength of invisible light, then the step of capturing a real-time image signal from the human body corresponding to the invisible light emitted by each light-emitting unit and reflected by the human body, and then the step of analyzing each real-time signal and defining a skin area according to the analysis result, and finally the step of calculating the change of reflected light intensity of the skin area to obtain a measured value corresponding to the physiological characteristic of the human body.

Preferably, the method further comprises the step of determining whether the measured value meets a reference range, and then giving off an alarm signal if the measured value exceeds the reference range.

Preferably, the invisible light emitted by the light-emitting units is near-infrared light.

Preferably, the physiological characteristic is the heartbeat of the human body.

Other advantages and features of the present invention will be fully understood by reference to the following specification in conjunction with the accompanying drawings, in which like reference signs denote like components of structure.

### DETAILED DESCRIPTION OF THE INVENTION

First of all, it should be noted that the present invention can be widely applied to different types of physiological detection or monitoring devices, and those skilled in the art can understand that the explanatory terms of the present preferred embodiment belong to a generic description that does not limit the application. For example, procedures, processes, or signal processing terms include, but are not limited to the description contents, do not limit the detection target, and do not limit the physiological characteristic that can be detected.

The application of the method for identifying and determining a physiological characteristic using invisible light in accordance with the present invention needs to use a plurality of light-emitting units capable of emitting invisible light, a signal processing unit and a digital image sensing unit. The light-emitting units can be configured to emit different wavelengths of invisible light. In the present preferred embodiment, the light-emitting units are formed of Near-Infrared LEDs for emitting invisible light of wavelength in the range of 850 nm to 1050 nm. Each light-emitting unit can be composed of a LED having a single wavelength, or a plurality of LEDs having the same wavelength. The light-emitting units are controlled to be individually and continuously activated so that only one light-emitting unit emits a single near-infrared light of the same wavelength to a human body to be detected at the same time. In the present preferred embodiment, the human body to be detected is an infant.

When the light-emitting units are separately and continuously controlled to emit invisible light to the human body to be detected, the digital image sensing unit capture the reflected light from the human body to be detected to generate a real-time image signal. To remove noise from the ambient light source for enhancing image quality, the digital image sensing unit can be activated to obtain the image signal of the detected human body in the ambient light as a basic signal before activating the light-emitting units, and the basic signal is than used for removing the ambient noise caused by the ambient light to the detected human body in the follow-up calculation. When a single light-emitting unit is activated, the digital image sensing unit is then synchronized to obtain the real-time image signal of the detected human body. In this way, the captured real-time image signals can be individually and one-to-one mapped to images reflected by the light-emitting units having a single and same wavelength.

After obtaining the real-time image signal formed by the reflected light, the digital image sensing unit may selectively remove the aforementioned basic signal generated by the ambient light source to the detected human body through the signal processing unit, and the real-time image signals formed by the light-emitting units having different wavelengths are processed and analyzed. If the analyzed image signal conforms to the reflection and absorption characteristics of human skin, it can be identified as a skin area according to the image distribution area, and then the reflected light intensity change of the skin area is calculated to obtain a measured value corresponding to the physiological characteristic of the human body to be detected. In this embodiment, the physiological characteristic is the heartbeat of the human body to be detected. Furthermore, it can be further determined whether the measured value is within a reference range. If the measured value exceeds the reference range, a warning signal is given to remind the parents and the caregiver.

In general, the present invention uses a plurality of light-emitting units of different wavelengths to emit invisible light separately and continuously toward the baby, and then uses an image sensing unit to capture the reflected light from the baby. Since emitting invisible light onto clothing, hair or skin surface can produce a different reflection power. When an invisible light is irradiated on the surface of a skin with high water content, a part of the invisible light will diffuse and reflect in the dermis layer of the skin, and the rest of the invisible light will penetrate the dermis and will be absorbed by the subcutaneous tissue layer. This reflection characteristic can be deduced from the Beer-Lambert's Law. The real-time image signal thus obtained can then be analyzed for defining the skin area. After the skin area is defined, calculate the change of reflected light intensity in the skin area to finally obtain a measured value corresponding to the physiological characteristic of the human body to be detected. Since the light absorption rate of arterial blood and skin tissue changes periodically, the physiological characteristic of the present preferred embodiment is based on the heartbeat (pulse) of the human body to be detected.

The present invention uses a small number of light-emitting units with different wavelengths to emit invisible light onto the human body to be detected for obtaining complete and sufficient image signals, thus, the overall cost is low. Further, by means of judging different characteristics of the human body surface to distinguish the skin area and the clothing area, the area and location for detecting physiological characteristics can be more accurately allocated to further obtain accurate physiological characteristics and better warning results.

What is claimed is:

1. A method for identifying and determining a physiological characteristic using invisible light, comprising the steps of:
a. respectively activating at least two light-emitting units to emit an invisible light onto a human body to be detected in such a manner that only one said light-emitting unit is activated at a time, each of said at least two light-emitting units being configured to emit a different wavelength of invisible light;
b. capturing a real-time image signal from said human body corresponding to the invisible light emitted by each said light-emitting unit and reflected by said human body;
c. analyzing each said real-time signal and defining a skin area according to the analysis result; and
d. calculating the change of reflected light intensity of said skin area to obtain a measured value corresponding to the physiological characteristic of said human body.

2. The method for identifying and determining a physiological characteristic using invisible light as claimed in claim 1, further comprising the step of: determining whether said measured value meets a reference range, and then giving off an alarm signal if said measured value exceeds said reference range.

3. The method for identifying and determining a physiological characteristic using invisible light as claimed in claim 1, wherein the invisible light emitted by said light-emitting units is near-infrared light.

4. The method for identifying and determining a physiological characteristic using invisible light as claimed in claim 1, wherein the wavelength of the invisible light emitted by said light-emitting units is within the range of 850 nm-1050 nm.

5. The method for identifying and determining a physiological characteristic using invisible light as claimed in claim 1, wherein said physiological characteristic is the heartbeat of the human body.
